(19) United States
(12) Patent Application Publication
(10) Pub. No.: US 2013/0137724 A1
(43) Pub. Date: May 30, 2013

(54) PHARMACEUTICAL COMPOSITIONS COMPRISING 7-(1H-IMIDAZOL-4-YLMETHYL)-5,6,7,8-TETRAHYDRO-QUINOLINE FOR RETINAL NEUROPROTECTION

(71) Applicant: ALLERGAN, INC., Irvine, CA (US)

(72) Inventors: Mohammed I. Dibas, Laguna Niguel, CA (US); John E. Donello, Dana Point, CA (US); Daniel W. Gil, Corona Del Mar, CA (US)

(73) Assignee: ALLERGAN, INC., Irvine, CA (US)

(21) Appl. No.: 13/684,954

(22) Filed: Nov. 26, 2012

Related U.S. Application Data
(60) Provisional application No. 61/563,886, filed on Nov. 28, 2011.

(51) Int. Cl. A61K 31/4709

(52) U.S. Cl. CPC A61K 31/4709
USPC 514/314

(57) ABSTRACT

The present invention relates to a method for retinal neuroprotection or for treating retinal diseases, in a patient in need thereof which comprises of administering a therapeutically effective amount of a pharmaceutical composition comprising a therapeutically effective amount of 7-(1H-Imidazol-4-ylmethyl)-5,6,7,8-tetrahydro-quinoline, its enantiomers or pharmaceutically acceptable salts thereof.

### RELATED APPLICATION

This application claims the benefit of U.S. Provisional Application Ser. No. 61/563,886, filed Nov. 28, 2011, the disclosure of which is hereby incorporated in its entirety herein by reference

### BACKGROUND OF THE INVENTION

The present invention relates to retinal neuroprotection in a patient in need thereof which comprises administering a therapeutically effective amount of a pharmaceutical composition comprising a therapeutically effective amount of 7-(1H-Imidazol-4-ylmethyl)-5,6,7,8-tetrahydro-quinoline or pharmaceutically acceptable salts thereof.

SUMMARY OF THE RELATED ART

Three alpha 1 and three alpha 2 adrenergic receptors have been characterized by molecular and pharmacological methods. Activation of these alpha 2 receptors evokes physiological responses and have useful therapeutic actions. Compound 7-(1H-imidazol-4-ylmethyl)-5,6,7,8-tetrahydro-quinoline is known as a potent alpha 2 adrenergic receptor pan agonist, activating all three alpha-2 receptor subtypes.

The racemic mixture and the two enantiomers of 7-(1H-imidazol-4-ylmethyl)-5,6,7,8-tetrahydro-quinoline are disclosed in U.S. Pat. No. 7,323,477 B2. U.S. Pat. No. 7,943,641 discloses a composition comprising (S)-(+)-7-(1H-Imidazol-4-ylmethyl)-5,6,7,8-tetrahydro-quinoline for the treatment of glaucoma or ocular hypertension. Binding studies showed that (S)-(+)-7-(1H-Imidazol-4-ylmethyl)-5,6,7,8-tetrahydro-quinoline, a partial agonist induces very little down-regulation of alpha 2A receptor. In contrast, brimonidine, a full agonist induced a strong reduction in the alpha 2A receptor density.

Brimonidine is compound (5-bromo-quinoxalin-6-yl)-imidazolidin-2-ylidene-amine, and the tartrate salt is sold under the trademark ALPHAGAN®P (available from Allergan, Inc.).

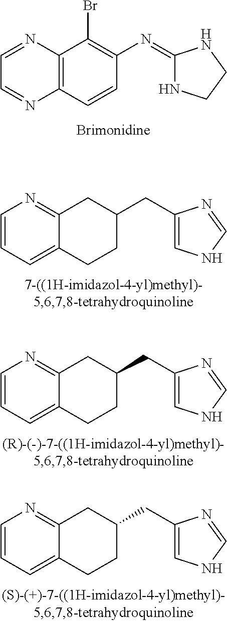 [SMILES: Brc1c(N=C2NCCN2)ccc2nccnc12 ; c1cnc2c(c1)CCC(Cc1c[nH]cn1)C2 ; c1cnc2c(c1)CC[C@H](Cc1c[nH]cn1)C2 ; c1cnc2c(c1)CC[C@@H](Cc1c[nH]cn1)C2]

### BRIEF SUMMARY OF THE INVENTION

The present invention provides pharmaceutical compositions, containing 7-(1H-Imidazol-4-ylmethyl)-5,6,7,8-tetrahydro-quinoline as active ingredient for modulating the alpha 2 adrenergic receptors. In another aspect the present invention provides pharmaceutical compositions, containing (S)-(+)-7-(1H-Imidazol-4-ylmethyl)-5,6,7,8-tetrahydro-quinoline as active ingredient for modulating the alpha 2 adrenergic receptors. In another aspect the present invention provides pharmaceutical compositions, containing (R)-(−)-7-(1H-Imidazol-4-ylmethyl)-5,6,7,8-tetrahydro-quinoline as active ingredient for modulating the alpha 2 adrenergic receptors. (S)-(+)-7-(1H-Imidazol-4-ylmethyl)-5,6,7,8-tetrahydro-quinoline is a potent alpha 2-adrenergic agonist, activating all three alpha 2 receptor subtypes. It is, however, a partial agonist of the alpha 2A receptor, which results in less receptor desensitization and down regulation (shown in table 2). This property is beneficial for sustained activity, particularly when the drug is delivered continuously.

We have now discovered that the pharmaceutical compositions of (S)-(+)-7-(1H-Imidazol-4-ylmethyl)-5,6,7,8-tetrahydro-quinoline are useful for retinal neuroprotection.

Thus, (S)-(+)-7-(1H-Imidazol-4-ylmethyl)-5,6,7,8-tetrahydro-quinoline is advantageous for neuroprotection in ocular diseases including but not limited to age related macular degeneration, wet macular degeneration, dry macular degeneration, geographic atrophy, diabetic retinopathy, diabetic macular edema, retinal vein occlusion, ocular hypertension, glaucoma, retinitis pigmentosa and neuritis secondary to multiple sclerosis. Our compound of interest is also useful for enhancing vision in patients with vision loss from conditions including ocular hypertension, glaucoma, retinitis pigmentosa and neuritis secondary to multiple sclerosis. This vision enhancement or retinal neuroenhancement can occur independent of neuroprotective effects of the compound.

“Vision loss”, as used here, means deficits in visual function including visual field, contrast sensitivity, night vision, color vision, acuity.

“Pharmaceutical composition,” as used here, means a composition that is suitable for administering to human patients for the treatment of disease. In one embodiment, therefore, the compound of the invention is formulated as pharmaceutically acceptable salts and further include one or more pharmaceutically acceptable excipients.

“Pharmaceutically acceptable salt” refers to those salts which retain the biological effectiveness and properties of the free base and which are obtained by reaction with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. (S)-(+)-7-(1H-Imidazol-4-ylmethyl)-5,6,7,8-tetrahydro-quinoline may be formulated with efficacy enhancing components as disclosed in U.S. Pat. No. 7,491,383 B2.

The (S)-(+)-7-(1H-Imidazol-4-ylmethyl)-5,6,7,8-tetrahydro-quinoline compound has physiochemical and pharmacokinetic properties that are beneficial for sustained activity, particularly when the drug is delivered continuously (e.g. to ocular implant).

The compound may be administered through different routes, including but not limited to topical eye drops, direct injection, application at the back of the eye or formulations that may further enhance the long duration of actions such as a slow releasing pellet, suspension, gel, solution, cream, ointment or sustained delivery devices such as any suitable drug delivery system (DDS) known in the art. While topical administration is preferred, this compound may also be used in an intraocular implant as described in U.S. Published Patent Application 20050244463. Such biocompatible intraocular implants include (S)-(+)-7-(1H-Imidazol-4-ylmethyl)-5,6,7,8-tetrahydro-quinoline and a polymer associated with (S)-(+)-7-(1H-Imidazol-4-ylmethyl)-5,6,7,8-tetrahydro-quinoline to facilitate release thereof into an eye for an extended period of time.

### BRIEF DESCRIPTION OF THE DRAWINGS

FIG. 1 shows that (S)-(+)-7-(1H-Imidazol-4-ylmethyl)-5,6,7,8-tetrahydro-quinoline reduced the damage caused by blue light.

### DETAILED DESCRIPTION OF THE INVENTION

In one aspect of the invention, there is provided a method for retinal neuroenhancement in a patient in need thereof which comprises, consists essentially of or consists of or consists of administering a therapeutically effective amount of a pharmaceutical composition comprising, consisting essentially of or consisting of a therapeutically effective amount of 7-(1H-Imidazol-4-ylmethyl)-5,6,7,8-tetrahydro-quinoline or pharmaceutically acceptable salts thereof.

In another aspect of the invention, there is provided a method for retinal neuroenhancement in a patient in need thereof which comprises, consists essentially of or consists of or consists of administering a therapeutically effective amount of a pharmaceutical composition comprising, consisting essentially of or consisting of a therapeutically effective amount of (R)-(−)-7-(1H-Imidazol-4-ylmethyl)-5,6,7,8-tetrahydro-quinoline or pharmaceutically acceptable salts thereof.

In another aspect of the invention, there is provided a method for retinal neuroenhancement in a patient in need thereof which comprises, consists essentially of or consists of or consists of administering a therapeutically effective amount of a pharmaceutical composition comprising, consisting essentially of or consisting of a therapeutically effective amount of (S)-(+)-7-(1H-Imidazol-4-ylmethyl)-5,6,7,8-tetrahydro-quinoline or pharmaceutically acceptable salts thereof.

In one aspect of the invention, there is provided a method for treating retinal diseases in a patient in need thereof which comprises, consists essentially of or consists of or consists of administering a therapeutically effective amount of a pharmaceutical composition comprising, consisting essentially of or consisting of a therapeutically effective amount of 7-(1H-Imidazol-4-ylmethyl)-5,6,7,8-tetrahydro-quinoline or the enantiomers thereof, or the tautomers thereof, or pharmaceutically acceptable salts thereof.

In another aspect of the invention, there is provided a method for treating retinal diseases in a patient in need thereof which comprises, consists essentially of or consists of or consists of administering a therapeutically effective amount of a pharmaceutical composition comprising, consisting essentially of or consisting of a therapeutically effective amount of (R)-(−)-7-(1H-Imidazol-4-ylmethyl)-5,6,7,8-tetrahydro-quinoline or the tautomers thereof, or pharmaceutically acceptable salts thereof. In another aspect of the invention, there is provided a method for treating retinal diseases in a patient in need thereof which comprises, consists essentially of or consists of or consists of administering a therapeutically effective amount of a pharmaceutical composition comprising, consisting essentially of or consisting of a therapeutically effective amount of (S)-(+)-7-(1H-Imidazol-4-ylmethyl)-5,6,7,8-tetrahydro-quinoline or the tautomers thereof, or pharmaceutically acceptable salts thereof.

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. It is intended that all such modifications will fall within the scope of the appended claims.

### EXAMPLE 1

### Visual Enhancement Model

Sixteen pigmented (Dutch-Belted) rabbits weighing 2-3 kg were used to evaluate the neuroenhancement effect of (S)-(+)-7-(1H-Imidazol-4-ylmethyl)-5,6,7,8-tetrahydro-quinoline. Rabbits were dosed with (S)-(+)-7-(1H-Imidazol-4-ylmethyl)-5,6,7,8-tetrahydro-quinoline through intravenous route. Spatial sweep visual evoked potential (sVEP) acuity. Spatial sVEP acuity was assessed with PowerDiva software version 1.8. Recordings were made bilaterally in conscious animals. The results demonstrate that (S)-(+)-7-(1H-Imidazol-4-ylmethyl)-5,6,7,8-tetrahydro-quinoline enhances visual acuity at 10-30 minutes post-dose in normal DB rabbits.

### EXAMPLE 2

### The Nerve Crush Model

This example describes the neuroprotective effect of (S)-(+)-7-(1H-Imidazol-4-ylmethyl)-5,6,7,8-tetrahydro-quinoline level in the rat nerve crush model. Sprague Dawley rats weighing 300-350 g were anesthetized with a mixture of ketamine (50 mg/kg) and xylazine (0.5 mg/kg). Lateral canthotomy was performed in the right eye and an incision was made in the superior conjunctiva adjacent to the rectus muscle. This was followed by a blunt dissection until optic nerve was exposed. A partial was applied to the optic nerve for 30 seconds, 2 to 3 mm distal from the globe, using calibrated cross-acting forceps. Care was taken not to interfere with retinal blood supply. (S)-(+)-7-(1H-Imidazol-4-ylmethyl)-5,6,7,8-tetrahydro-quinoline was administered at 0.03, 0.1, 0.3, 1 mg/kg SC two hours before nerve injury, the vehicle PBS was administered SC as a negative control whereas brimonidine 0.1 mg/kg was given by IP injection as a positive control. Control animals received phosphate-buffered saline (PBS) vehicle. The experiment was terminated 12-15 days later.

### EXAMPLE 3

### The Chronic Ocular Hypertension Model

Intraocular Pressure (IOP) was elevated in male Witar rats weighing 350-450 g using laser photocoagulation with blue-green argon laser (Coherent, Palo Alto, Calif.). Rats were anesthetized with a mixture of ketamine (15 mg/kg), acepromazine (1.5 mg/kg), and xylazine (0.3 mg/kg). Laser treatment was done in two parts (1-week interval) on limbal and epsiscleral veins. The amount of energy used was 1 W for 0.2 seconds, delivering a total of 150 spots (50-100 μM). Intraocular pressure was measured using tonometer (TONO-PEN: mentor, Norwell, Mass.). Rats were sedated with 3.0 mg/kg IM acepromazine during IOP measurements. Proparacaine 0.5% was applied topically on the eyes to anesthetize the cornea. Initial IOP measurements were done before laser treatment to determine baseline IOP and subsequent measurements were done once a week.

(S)-(+)-7-(1H-Imidazol-4-ylmethyl)-5,6,7,8-tetrahydro-quinoline was administered constantly using an osmotic pump (Alzet Osmotic Pumps, Duret Corp., Cupertino, Calif.) which was inserted subcutaneously on the back at 0.03, 0.1, 0.3, 1 mg/kg SC two hours before nerve injury, the vehicle PBS was administered SC as a negative control whereas brimonidine 0.1 mg/kg was given by IP injection as a positive control. Control animals received phosphate-buffered saline (PBS) vehicle. The experiment was terminated 12-15 days later.

### EXAMPLE 4

### Membrane [Methyl-³H] Rauwolscine Saturation Binding

Results from an in vitro GTPase assay of alpha 2A receptor activation are shown in Table 1 for brimonidine and (S)-(+)-7-(1H-Imidazol-4-ylmethyl)-5,6,7,8-tetrahydro-quinoline. The data show that (S)-(+)-7-(1H-Imidazol-4-ylmethyl)-5,6,7,8-tetrahydro-quinoline is a partial agonist with level of efficacy 0.6 of the efficacy of the full agonist brimonidine.

TABLE 1

| Compound | EC50 (nM) | Relative Efficacy |
|---|---|---|
| Brimonidine | 10 | 1.0 |
| (S)-(+)-7-(1H-Imidazol-4-ylmethyl)- | <1 | 0.6 |
| 5,6,7,8-tetrahydro-quinoline | | |

In the GTPase assay, receptor activation results in dissociation of GDP and binding of the nonhydrolyzable [³⁵S]GTPγS to receptor-coupled G-proteins. The extent of binding is a measure of receptor activation. Membranes were prepared from HEK cells transiently expressing the alpha2A receptor and the three subunits of the Gᵢ G-protein. Membranes were thawed and resuspended using a Polytron disrupter in 4° C. membrane buffer and quantified via Bradford assay. The quantified protein was then added to reaction buffer [50 mM Tris-HCl, 100 mM NaCl, 5 mM MgCl₂, 1 mM DTT, 1 mM EDTA, 3 μM propranolol, and 0.1 mM AMP; pH 7.4 and 4° C.] to achieve a 100 μg/mL concentration, allowed to incubate for 15 minutes, and then incubated for an additional 10 minutes in the presence of 6 μM GDP. The above mixture was then aliquoted, 50 μL/well, in a 96 well plate, combined with an equal volume of test compound dissolved in assay buffer [50 mM Tris-HCl, 100 mM NaCl, 5 mM MgCl₂, 1 mM EDTA, and 1 mM DTT, pH 7.4 and 4° C.], and allowed to incubate for 5 minutes. Immediately following the above incubation, the mixture was combined with 50 μL of 1.5 nM [³⁵S]GTPγS in assay buffer and shaken covered for 60 minutes at 25° C. Assays were terminated by vacuum filtration over GF/B filters blocked with 0.5% BSA. Filters were then washed with 4° C. wash buffer [50 mM Tris-HCl, 100 mM NaCl, and 5 mM MgCl₂, pH 7.5]. The incorporated [³⁵S]GTPγS was determined using a Microbeta 1450B liquid scintillation counter after the plates had dried overnight.

Human embryonic kidney (HEK) 293T cells stably transfected with the α_{2A}-adrenergic receptor were grown to 50-60% confluency on T-175 culture flasks in DMEM (Gibco, cat. #11995), 10% FBS (Gibco, cat. #16140), 0.25 ug/mL puromycin (Sigma, cat. #P-8833), and 1% antibiotic-antimycotic (Gibco, cat. #15240) maintained at 37° C. and 5% CO₂. After reaching the desired confluency, the cells were incubated for 24 hours in growth media treated with 0, 1,000 nM brimonidine and (S)-(+)-7-(1H-Imidazol-4-ylmethyl)-5,6,7,8-tetrahydro-quinoline and maintained at 37° C. and 5% CO₂. The cells were then washed with room temperature Dulbecco's phosphate buffered saline (DPBS; Gibco, cat. #14190).

TABLE 2

Percent of Bₘₐₓ Decrease
Compound

| | | (S)-(+)-7-(1H-Imidazol-4- |
|---|---|---|
| | | ylmethyl)-5,6,7,8-tetrahydro- |
| Concn (μM) | Brimonidine | quinoline |
| 1 | −45 | −25 |

Cells were harvested with 4° C. Tris-EDTA buffer [50 mM Tris-HCl, 5 mM EDTA; pH 7.4] and centrifuged at 5,000×g for 5 minutes at 4° C. Membrane preparation was conducted by resuspending the cell pellets in 4° C. Tris-EDTA buffer and lysed with a Polytron disrupter two times (setting 7, 5 seconds each). The lysed suspension was then centrifuged at ˜35,000×g for 32 minutes at 4° C. After decanting the supernatant, the pelleted material was further lysed in 4° C. Tris buffer [50 mM Tris-HCl; pH 8.0] using the Polytron disrupter (setting 4, 5 seconds). Membranes were then aliquoted, pelleted at ˜37,000×g for 32 minutes at 4° C., and stored at −80° C. until use.

Membranes were thawed and resuspended using a Polytron disrupter in 4° C. HBSS-HEPES buffer [1 part 1M HEPES: 5 parts 10× Hank's Balanced Salt Solution: 43.8 parts H₂O; pH 7.4 with KOH] and quantified via Bradford assay. The quantified protein was further diluted with HBSS-HEPES buffer to achieve a 100 μg/mL concentration. Membrane suspension was plated in a 96-well plate at 200 μLs/well ±10 μM phentolamine HCl (Sigma, cat. #P-7547) and [Methyl-³H] Rauwolscine (15 nM to 0.05 nM; PerkinElmer, cat. #NET722250UC). The assay plate was slowly shaken and incubated at 25° C. for ninety minutes. Immediately following the above incubation, the assays were terminated by vacuum filtration over GF/B filters. Filters were then washed with 4° C. HBSS-HEPES buffer. The incorporated [Methyl-³H] Rauwolscine was determined using a Microbeta 1450B liquid scintillation counter after the plates had dried overnight.

Binding studies showed that (S)-(+)-7-(1H-Imidazol-4-ylmethyl)-5,6,7,8-tetrahydro-quinoline, a partial agonist, induces very little down-regulation of alpha2A receptor. In contrast, brimonidine, a full agonist induced a strong reduction in the alpha 2A receptor density.

### EXAMPLE 5

### The Blue Light Model of Retinal Degeneration in Rats

In order to demonstrate the advantage of partial alpha 2A agonists in treatment of retinal disease, (S)-(+)-7-(1H-Imidazol-4-ylmethyl)-5,6,7,8-tetrahydro-quinoline was compared the vehicle in the blue light model of retinal degeneration in rats. (S)-(+)-7-(1H-Imidazol-4-ylmethyl)-5,6,7,8-tetrahydro-quinoline was administered continuously with a subcutaneous pump at a dose of 0.3 mg/kg/day and 1 mg/kg/day, respectively starting two days before blue light exposure. These concentrations result in drug levels in the retina of 1.3 ng/g (S)-(+)-7-(1H-Imidazol-4-ylmethyl)-5,6,7,8-tetrahydro-quinoline, which are sufficient for pharmacological activity. Twenty 4-month old male Sprague-Dawley rats (body weight 470-550 g) were used in this study. The animals were exposed to room light on a 12 hour light/12 hour dark cycle before the experiment. All animals were dark adapted overnight (16-20 hours) before blue light. Under the intensity of 6100-6500 lux, rats were exposed to blue light for 4 hours. After the blue light, rats were placed in the dark for another 3 days before returning to normal 12 hour light/12 hour dark. Ocular Coherence Tomography (OCT) measurement was performed at 7 days post blue light exposure.

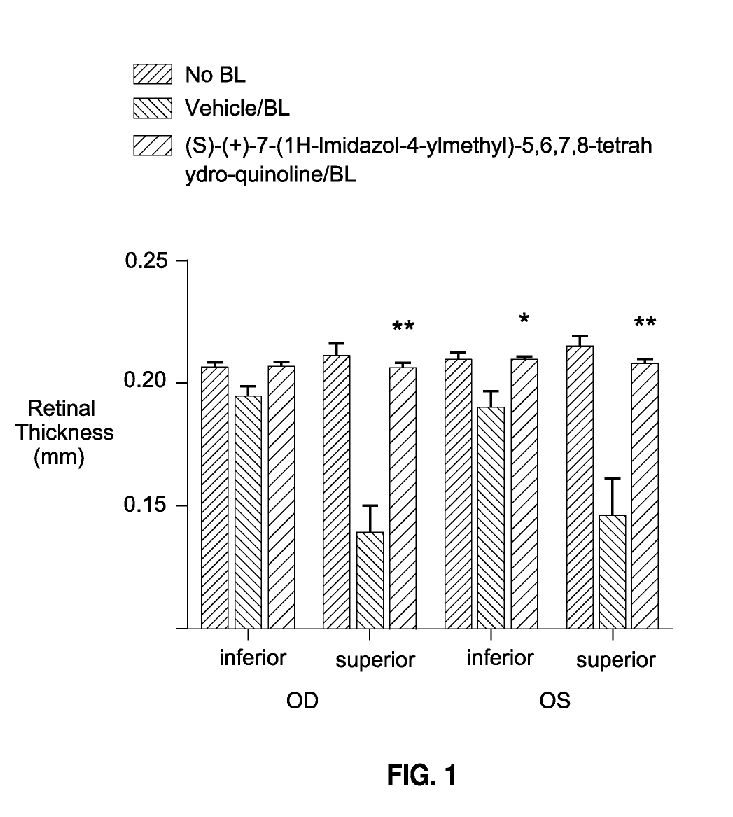

The results, FIG. 1, demonstrate that blue light exposure with just saline treatment leads to dramatic reduction of retinal thickness measured by OCT, particularly in the superior retina. Histology studies have shown that the reduction in retinal thickness is attributable to loss of photoreceptors. Treatment with (S)-(+)-7-(1H-Imidazol-4-ylmethyl)-5,6,7,8-tetrahydro-quinoline significantly reduced the damage caused by blue light.

What is claimed is:

1. A method for retinal neuroprotection in a patient, which comprises treating said patient with an effective amount of a pharmaceutical composition comprising 7-(1H-Imidazol-4-ylmethyl)-5,6,7,8-tetrahydro-quinoline or enantiomers or a salt thereof.

2. A method according to claim 1, wherein the pharmaceutical composition comprises an effective amount of (S)-(+)-7-(1H-Imidazol-4-ylmethyl)-5,6,7,8-tetrahydro-quinoline or a salt thereof.

3. A method according to claim 1, wherein the pharmaceutical composition comprises an effective amount of (R)-(−)-7-(1H-Imidazol-4-ylmethyl)-5,6,7,8-tetrahydro-quinoline or a salt thereof.

4. A method for retinal neuroprotection according to claim 1, wherein the disease is selected from: age related macular degeneration, wet macular degeneration, dry macular degeneration, geographic atrophy, diabetic retinopathy, diabetic macular edema, retinal vein occlusion, ocular hypertension, glaucoma, retinitis pigmentosa and neuritis secondary to multiple sclerosis.

5. An article of manufacture comprising packaging material and a pharmaceutical agent contained within said packaging material, wherein the pharmaceutical agent is therapeutically effective for treating a retinal disease and wherein the packaging material comprises a label which indicates the pharmaceutical agent can be used for treating a retinal disease and wherein said pharmaceutical agent comprises an effective amount of (S)-(+)-7-(1H-Imidazol-4-ylmethyl)-5,6,7,8-tetrahydro-quinoline.

6. A method for treating retinal diseases in a patient suffering thereof, which comprises treating said patient with an effective amount of a pharmaceutical composition comprising 7-(1H-Imidazol-4-ylmethyl)-5,6,7,8-tetrahydro-quinoline or the enantiomers thereof, or the tautomers thereof, or pharmaceutically acceptable salts thereof.

7. A method for treating retinal diseases according to claim 6, wherein the pharmaceutical composition comprises an effective amount of (S)-(+)-7-(1H-Imidazol-4-ylmethyl)-5,6,7,8-tetrahydro-quinoline or the tautomers thereof, or pharmaceutically acceptable salts thereof.

8. A method for treating retinal diseases according to claim 6, wherein the pharmaceutical composition comprises an effective amount of (R)-(−)-7-(1H-Imidazol-4-ylmethyl)-5,6,7,8-tetrahydro-quinoline or the tautomers thereof, or pharmaceutically acceptable salts thereof.

9. A method according to claim 6, wherein the disease is selected from: age related macular degeneration, wet macular degeneration, dry macular degeneration, geographic atrophy, diabetic retinopathy, diabetic macular edema, retinal vein occlusion, ocular hypertension, glaucoma, retinitis pigmentosa and neuritis secondary to multiple sclerosis

10. A method for enhancing vision in patients with vision loss from conditions including ocular hypertension, glaucoma, retinitis pigmentosa and neuritis secondary to multiple sclerosis by administering a pharmaceutical composition comprising an effective amount of (S)-(+)-7-(1H-Imidazol-4-ylmethyl)-5,6,7,8-tetrahydro-quinoline or the tautomers thereof, or pharmaceutically acceptable salts thereof.

11. An article of manufacture comprising packaging material and a pharmaceutical agent contained within said packaging material, wherein the pharmaceutical agent is therapeutically effective for treating a retinal disease and wherein the packaging material comprises a label which indicates the pharmaceutical agent can be used for treating a retinal disease and wherein said pharmaceutical agent comprises an effective amount of 7-(1H-Imidazol-4-ylmethyl)-5,6,7,8-tetrahydro-quinoline or the enantiomers thereof, or the tautomers thereof.

12. An article of manufacture according to claim 11, wherein said pharmaceutical agent comprises an effective amount of (S)-(+)-7-(1H-Imidazol-4-ylmethyl)-5,6,7,8-tetrahydro-quinoline or the tautomers thereof.

13. An article of manufacture according to claim 11, wherein said pharmaceutical agent comprises an effective amount of (R)-(−)-7-(1H-Imidazol-4-ylmethyl)-5,6,7,8-tetrahydro-quinoline or the tautomers thereof.
